(19) United States
(12) Patent Application Publication
(10) Pub. No.: US 2023/0181386 A1
(43) Pub. Date: Jun. 15, 2023

(54) MALE DISPOSABLE UNDERWEAR WITH FLY

(71) Applicant: Mmoja Ajabu, Indianapolis, IN (US)

(72) Inventor: Mmoja Ajabu, Indianapolis, IN (US)

(21) Appl. No.: 17/547,447

(22) Filed: Dec. 10, 2021

(51) Int. Cl. A61F 13/496; A61F 13/491; A61F 13/49; A41B 9/00

(52) U.S. Cl. CPC A61F 13/496; A41B 9/005; A61F 13/4915; A61F 13/49011; A61F 13/49014; A61F 2013/8408; A61F 2013/49098

(57) ABSTRACT

A liquid-absorbent disposable male brief, including a brief defining an elastic waistband, a pair of elastic leg bands, and an enclosure extending there between, a fly formed through the enclosure and positioned between the leg bands and defining a first flap and a second flap, a liquid absorbent pad positioned within the enclosure and secured thereto, and at least a first and a second liquid absorbent fly flap liner, each respective fly flap liner operationally connected to a respective flap.

### CROSS REFERENCE TO RELATED APPLICATIONS

This utility patent application is a continuation-in-part of, and claims priority to, co-pending U.S. Pat. Application Serial No. 16/5787,756, filed on Sep. 30, 2019, which claimed priority to then co-pending U.S. Provisional Pat. Application 62/739,481, filed Oct. 1, 2018, which is incorporated herein by reference.

### TECHNICAL FIELD

The present novel technology relates to a male disposable incontinence underwear, and more particularly, to a male disposable incontinence underwear that includes an absorbent, reinforced vertical aperture, such as a fly, situated in the front of the underwear.

### BACKGROUND

Many male disposable incontinence underwear designs are known in the prior art. However, most assume that the wearer always requires the services of the incontinence underwear, and thus provide no means for urination other than pulling the underwear down. One known design provides a zipper fly, but zippers in disposable underwear are inherently cheap and flimsy and suffer the problem of not providing a liquid-proof seal for when incontinence is experienced. Thus, there remains a need for improved male incontinence underwear. The present novel technology addresses this need.

### SUMMARY

The present novel technology provides a pull up style male disposable brief that combines the capture and absorbency features of incontinence underwear in general with a relatively liquid-tight vertical aperture, such as a fly, through which the wearer may urinate in the normal fashion by opening the aperture to urinate outside the underwear.

This more important features of the present male disposable incontinence underwear have been broadly outlined herein so that the detailed description thereof that follows may be better understood and in order that the present contribution to the art may be better appreciated.

Objects of the present male disposable incontinence underwear, along with various novel features that characterize the novel technology are particularly pointed out in the claims forming a part of this disclosure. For better understanding of the male disposable incontinence underwear, its operating advantages and specific objects attained by its uses, refer to the accompanying drawings and description.

In one aspect of the novel technology, a reusable absorbent device for incontinent males is provided.

It is further an object of the novel technology to provide underwear in the pull-up style which is secure around the legs and waist of the user, yet which also allows the front of the underwear to be opened for urination therethrough and then reclosed.

Another object of the present novel technology is to provide a resealable fly that provides a liquid-tight barrier to urination experienced within the confines of the underwear.

The above and other objects of the novel technology are realized in an illustrative embodiment of an underwear which advantageously includes an inner water permeable layer, an outer nonabsorbent layer, and an absorbent padding positioned between the inner and outer layers.

In one embodiment of the novel technology the underwear contains an opening, which extends from a waist section through a front portion and into a crotch portion thereof, which has overlapping flaps which are sealed in a closed position by a reusable hook-and-loop style fastener. The flaps are separable to form a penile opening and are reclosable and resealable.

The reusable fastener may be attached to the outer flap and to the outer surface of the front portion of the underwear. The fastener may extend along a portion of the edge of the outer flap or may extend along the entire length of the outer flap entirely sealing the edge thereof to the front portion of the underwear.

Thevertical aperture, such as a fly, has been configured to be impervious to bodily fluids when closed, thereby maintaining the purposefulness of the underwear for treating incontinence, while allowing for directed urination as preferred. The vertical aperture consists of a series of hook and loop fasteners. Typically, the hooks and/or the loops are made of a liquid absorbent material, such as a super-absorbent polymer, and both absorb liquid (such as urine) upon contact and swell with absorbed urine to eliminate gaps therebetween and make a liquid-tight seal. When closed, the underwear functions to absorb and capture any discharge of bodily fluids thereby protecting the outer garments of the wearer from unintentional soiling. When the aperture is opened, however, the penis of the wearer may be accessed therethrough and urination may occur in the normal fashion, directed away from the wearer and his clothes. As such, the male disposable incontinence underwear here presented is a device convenient for men suffering from incontinence who are still active in day to day society. The need to undo and remove an outerwear without the aforementioned aperture, in the hopes of urinating intentionally, can be embarrassing and cumbersome. Furthermore, the delay caused in removing such an outerwear may result in the occurrence of an unintentional discharge, precipitating the very outcome the underwear was designed to protect against. Therefore, the present novel technology has been devised to allow an incontinent male relative freedom to interact normally with society as preferred while protecting against the unintentional discharges that affect incontinent males.

The present male disposable incontinence underwear can be configured to various sizes appropriate for males of different ages. The present device is therefore useful for potty training young adults and toddlers who are learning how to urinate in the normal fashion, standing erect and consciously directing their urine stream, while protecting against the unintentional discharge of bodily fluids which may also result.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

Unless otherwise stated, a reference to a compound or component includes the compound or component by itself, as well as in combination with other compounds or components, such as mixtures of compounds.

As used herein, the singular forms “a”, “an” and “the” include the plural reference unless the context clearly dictates otherwise.

All publications, patents and patent applications cited herein, whether supra or infra, are hereby incorporated by reference in their entirety to the same extent as if each individual publication, patent or patent application was specifically and individually indicated to be incorporated by reference.

FIG. 1 displays the male underwear with the front fastener, such as a fly, open.

FIG. 2 displays a close-up view of the fly or front fastener on the male underwear.

FIG. 3 displays a front view of the fly or front fastener on the male underwear.

FIG. 4 displays the male underwear on a user.

FIG. 5 displays a front view of an alternative embodiment of the fly or front fastener on the male underwear.

FIG. 6 displays another front view of the alternative embodiment of the male underwear of FIG. 5 illustrating that the fly does not open all the way to the waistband.

FIG. 6A is an enlarged partial view of the fly flap hook and loop connectors.

FIG. 7 is front view of a second embodiment male underwear with the front fastener, such as a fly, open.

FIG. 8 displays a close-up view of the fly or front fastener on the male underwear.

FIG. 9 displays a front view of the fly or front fastener on the male underwear.

FIG. 10 displays a first perspective view of the male underwear of FIG. 9.

FIG. 11 displays a second perspective view of the male underwear of FIG. 9.

FIG. 12 displays an enlarged exploded view of the multiple plys of the underwear of FIG. 7.

Corresponding reference characters indicate corresponding parts throughout the several views. Although the drawings represent embodiments of the present disclosure, the drawings are not necessarily to scale and certain features may be exaggerated in order to better illustrate and explain the present disclosure.

### DETAILED DESCRIPTION OF THE EXEMPLARY EMBODIMENTS

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

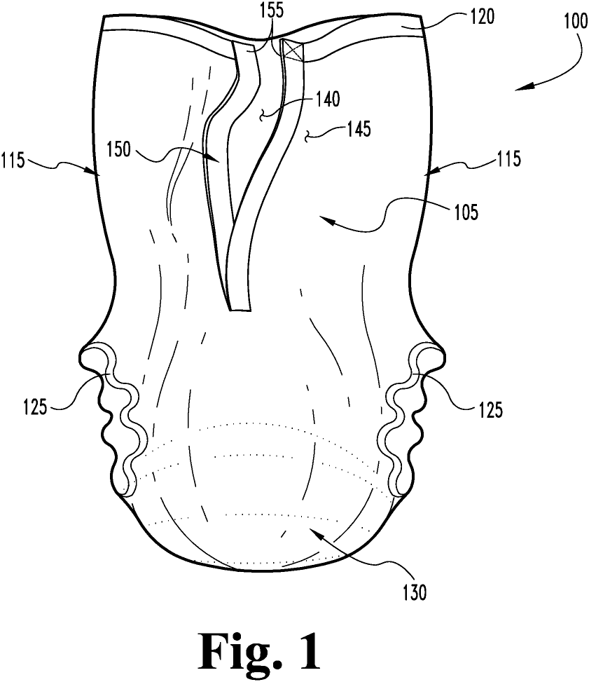

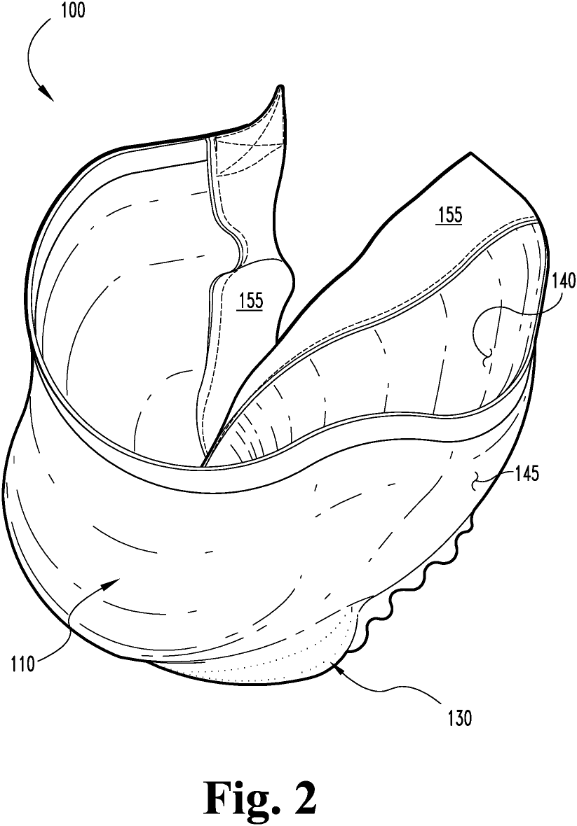

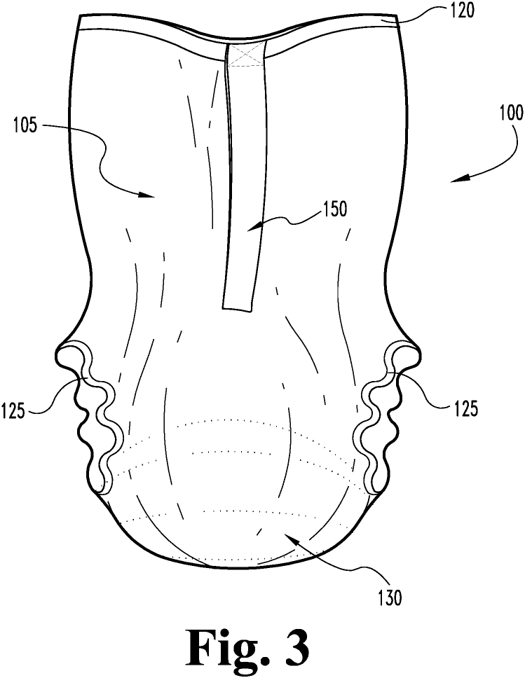

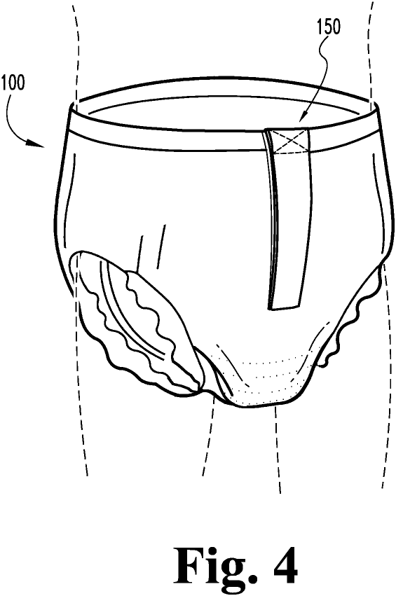

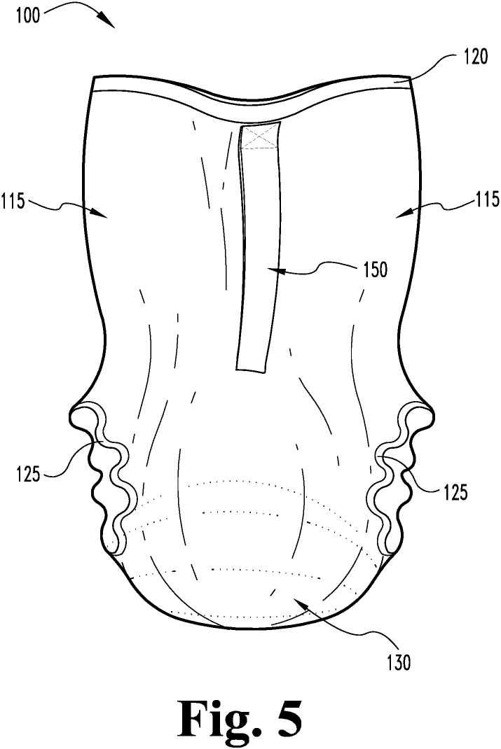

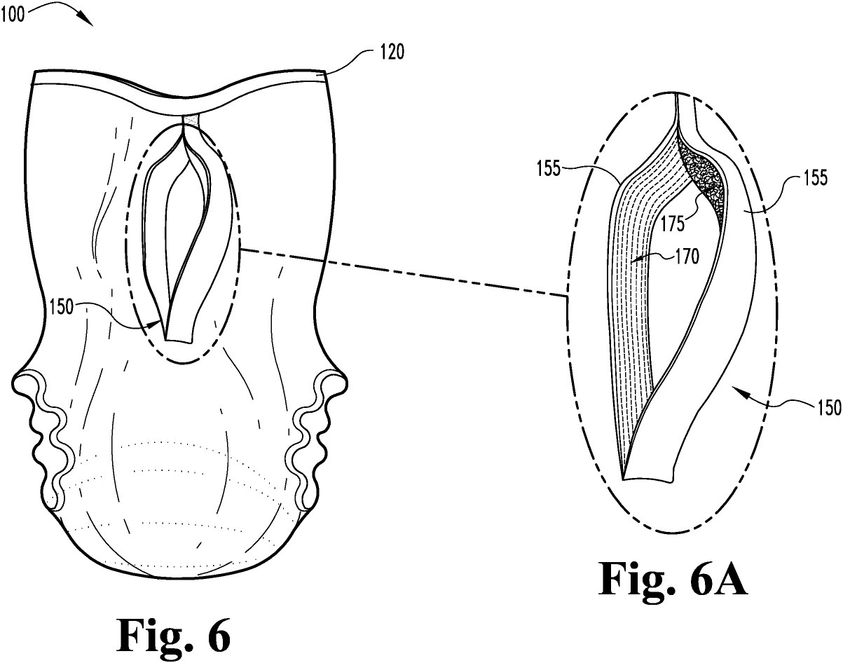

FIG. 1-6B illustrate a first embodiment male incontinence underwear 100 including a front portion 105, a back or rear portion 110, side portions 115, an upper elastic waistband portion 120, elastic leg band portions 125, and a crotch portion 130. The front and back portions 105, 110 are continuous with each of the side portions 115, in the pull-up style of underwear. The front, back, and side portions 105, 110, 115 are comprised of a material that is typically gathered and stitched to permit stretching to variably conform and to accommodate the waist size of the user. The crotch portion 130 typically includes additional adsorbent material between the (typically liquid absorbent) inner 140 and (typically liquid impermeable) outer 145 layers and gatherings about the legs to prevent leakage.

A fly portion or assembly 150 is typically positioned on and through the front portion 105, more typically extending into the crotch portion 130, and typically includes two or more sealingly engageable flaps 155. More typically, at least one of the fly flaps 155 includes a plurality of hooks 170 disposed thereupon, and at least one other of the fly flaps 155 includes a plurality of loops 175 disposed thereupon, wherein the hooks and loops 170, 175 are lockingly engageable when pressed together and lockingly disengeagable when pulled apart with sufficient force, so as to define a repeatedly lockingly closeable/openable or engageable/disengageable fly member 150.

More typically, at least one of the plurality of hooks 170 and the plurality of loops 175 is made of a liquid absorbent polymer or like material, such that when sealed or lockingly engaged, the fly assembly 150 actively absorbs liquid incumbent thereupon and exposure to liquid urges lockingly engaged hooks and loops 170, 175 to expand into one another, blocking any openings that may be present and enhancing the liquid-tightness of the sealed fly 150.

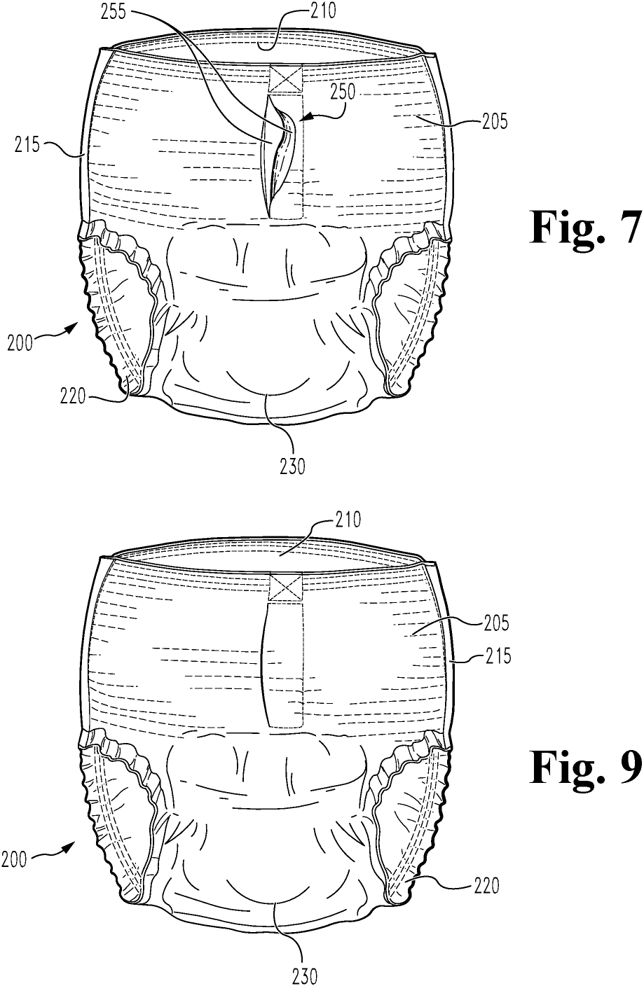

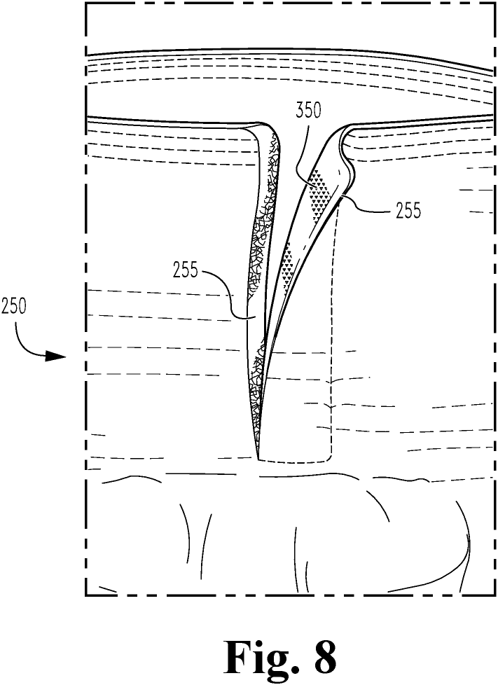

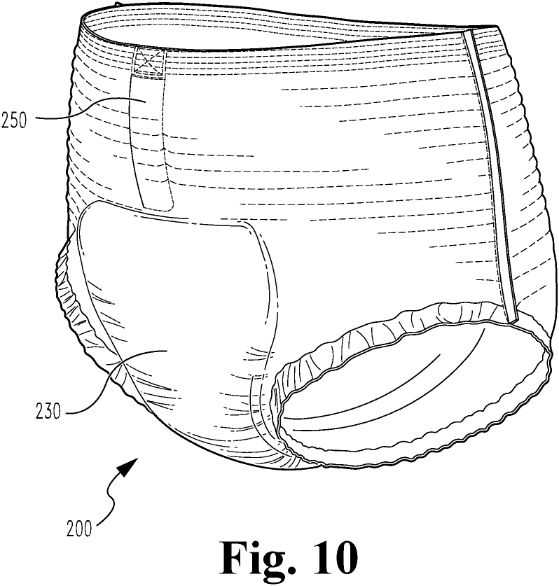

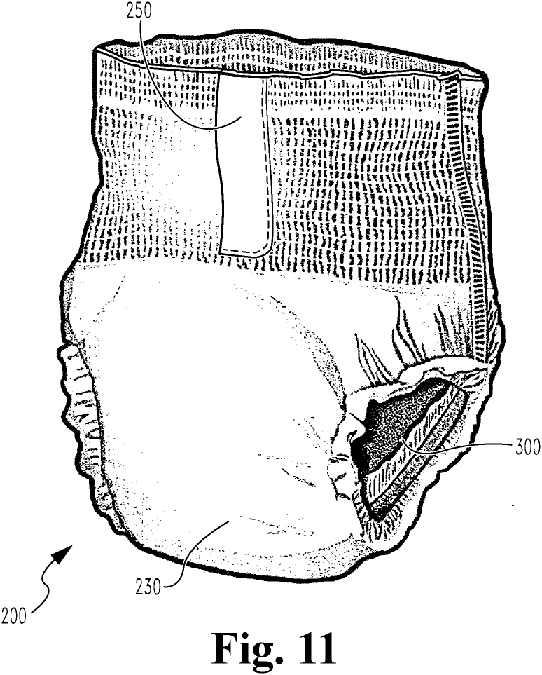

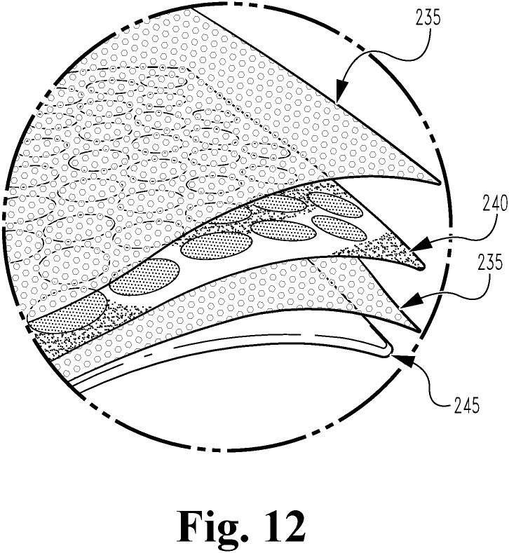

In another embodiment, as shown in FIGS. 7-12, the male incontinence underwear 200 is comprised of a front portion 205, a back or rear portion 210, side portions 215, an upper elastic waistband portion 220, elastic leg band portions 225, and a crotch portion 230. These portions are contiguous with one another and are made of a thin, lightweight material and are typically ruched so as to provide some stretch and comfort to the wearer. The front and back portions 205, 210 are continuous with each of the side portions 215, in the pull-up style of underwear and may define a single layer of material or multiple layers, including a liquid impermeable layer 245 and an absorbent, liquid permeable layer 240. The absorbent layer 240 may be woven or non-woven and be odor and liquid absorbent. The front, back, and side portions 205, 210, 215 are comprised of a material that is typically gathered and stitched to permit stretching to variably conform and to accommodate the waist size of the user. The underwear 200 further comprises a liquid absorbent liner 300 extending from the front portion 205 to the back portion 210 and over the intervening crotch portion 230. The liner 300 is typically made of a liquid adsorbent material, such as cotton, to temporarily hold excess liquid and to prevent leakage.

A fly portion or assembly 250 is typically positioned on and through the front portion 105, more typically extending into the crotch portion 230, and typically includes two flaps 255. More typically, at least one of the fly flaps 255 includes an absorbent fly liner 350 disposed thereupon, and more typically both flaps include a fly liner disposed thereupon. The fly liner 350 is likewise made of a liquid absorbent material.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

1. A disposable, liquid-absorbent male brief comprising:
a front portion;
a rear portion;
a crotch portion disposed between the front and rear portions;
an absorbent liner extending from the front to the rear portion;
a fly portion disposed in the front portion defining overlapping flaps; and
a fly liner portion defining respective liquid absorbent portions covering respective flaps.

2. The disposable, liquid-absorbent male brief of claim 1, wherein the front portion, rear portion, and fly portion are made of a liquid-impermeable material and is used in the pull-up style.

3. The disposable, liquid-absorbent male brief of claim 1, comprising of an interior surface and an oppositely disposed exterior surface; wherein the interior surface is in continuous contact with the wearer.

4. The disposable, liquid-absorbent male brief of claim 1 wherein the fly portion is made up respective front portion, rear portion, and crotch portion each define a respective liquid impermeable layer and a liquid permeable layer.

5. The disposable, liquid-absorbent male brief of claim 1, and further comprising of an inner water permeable layer, an outer nonabsorbent layer, and a liquid-absorbent padding position between the inner and outer layers.

6. A liquid-absorbent disposable male brief, comprising
a brief defining an elastic waistband, a pair of elastic leg bands, and an enclosure extending there between;
a fly formed through the enclosure and positioned between the leg bands and defining a first flap and a second flap;
a liquid absorbent pad positioned within the enclosure and secured thereto; and
at least a first and a second liquid absorbent fly flap liner, each respective fly flap liner operationally connected to a respective flap.

7. The absorbent male brief of claim 6, wherein the first fly flap liner is attached to the exterior surface of the front portion; wherein the second fly flap liner is attached to the interior surface of the front portion.

8. The absorbent male brief of claim 6, wherein the enclosure is a liquid and odor absorbent material.

9. The absorbent male brief of claim 6, wherein the enclosure is nonwoven.

10. The absorbent male brief of claim 6, wherein the enclosure includes a liquid impermeable layer.

11. The absorbent male brief of claim 6, wherein the respective liquid absorbent fly flap liners and liquid absorbent pad are made of cotton.
